(19) United States
(12) Patent Application Publication
(10) Pub. No.: US 2016/0366880 A1
(43) Pub. Date: Dec. 22, 2016

(54) ANTIMICROBIAL DEVICES COMPRISING POLYURETHANE, AND RELATED METHODS AND ASSEMBLIES

(71) Applicant: Health Line International Corp., Salt Lake City, UT (US)

(72) Inventors: Joel K. Faulkner, Salt Lake City, UT (US); Aaron Garcia Faulkner, Salt Lake City, UT (US); Filemon Martinez, Clinton, UT (US); Li Min She, Foshan (CN)

(73) Assignee: Health Line International Corp., Salt Lake City, UT (US)

(21) Appl. No.: 15/189,831

(22) Filed: Jun. 22, 2016

Related U.S. Application Data
(60) Provisional application No. 62/183,125, filed on Jun. 22, 2015.

(51) Int. Cl. A01N 25/34; A01N 59/16; A61L 2/23; A01N 47/10

(52) U.S. Cl. CPC A01N 25/34; A01N 47/10; A01N 59/16; A61L 2/23; A61L 2202/24

(57) ABSTRACT

An antimicrobial device comprises a housing structure configured to couple to a medical device and comprising an antimicrobial polymer material comprising polyurethane, the antimicrobial polymer material formulated to at least partially decontaminate surfaces of the medical device in at least temporary physical contact with the housing structure. A medical device assembly, and a method of decontaminating a medical device are also described.

### CROSS-REFERENCE TO RELATED APPLICATION

This application claims the benefit of U.S. Provisional Patent Application Ser. No. 62/183,125, filed Jun. 22, 2015, the disclosure of which is hereby incorporated herein in its entirety by this reference.

### FIELD

The disclosure generally relates to antimicrobial devices for use with medical devices, and to related medical device assemblies and methods. In particular, embodiments of the disclosure relate to antimicrobial devices formed of and including an antimicrobial polymer material formulated to decontaminate portions of one or more medical device(s) in physical contact therewith.

### BACKGROUND

In the medical field and, in particular, within the area of delivery of fluids to a subject (e.g., patient) and/or removal of fluids from a subject, a need exists to prevent the transmission of pathogens (e.g., microorganisms, bacteria, viruses, fungi, etc.) into or onto the subject from a potentially contaminated surface of a medical device. For example, bloodstream infections, which may be caused by pathogens that enter patients by way of intravascular (IV) catheters, are a significant cause of illness and excess medical costs and may result in serious infection and/or death.

Some approaches to reducing the likelihood of infection and/or death resulting from a contaminated surface of a medical device can include treating the surface of the medical device with at least one of a liquid antimicrobial agent and radiation. For example, an disinfectant-impregnated swab (e.g., a swab infiltrated with at least one of an alcohol, such as isopropyl alcohol and/or ethanol; hypochlorite; an oxygen reactive species, such as hydrogen peroxide; Triclosan; etc.) can be wiped across one or more surface(s) of the medical device by a healthcare provider so that as the alcohol applied by the swab evaporates, pathogens on the surface(s) of the medical device are destroyed. As another example, one or more surface(s) of the medical device may be exposed to at least one of ultraviolet (UV) radiation and gamma radiation to destroy pathogens thereon. However, such approaches can be time consuming, costly, and prone to human error. In addition, such approaches and can also be insufficient to adequately protect the medical device from future contamination (e.g., over prolonged periods of time prior to the use of the medical device), requiring frequent repetition of the process and excessive use of disinfectants (e.g., Triclosan), which can have undesirable environmental impacts.

Additional approaches toward reducing the likelihood of infection and/or death resulting from a contaminated surface of a medical device can include impregnating and/or coating the medical devices structure itself with one or more antimicrobial agent(s) (e.g., biocides). For example, the medical device may be impregnated (e.g., doped, infiltrated, etc.) and/or coated with one or more silver-based antimicrobial agent(s) and/or one or more non-silver-based antimicrobial agent(s) to reduce the likelihood of pathogenic contamination of the medical device. However, the effectivity of such approaches can diminish or even terminate over time as the antimicrobial agent(s) become exhausted. In addition, such approaches can often be insufficient to adequately protect the medical device against a variety of pathogens, such as pathogens resistant to the antimicrobial agent(s) impregnated within and/or coated on the medical devices structure.

Further approaches toward reducing the likelihood of infection and/or death resulting from a contaminated surface of a medical device can include decontaminating the medical device using one or more other medical device(s) (e.g., medical cap(s)) having antimicrobial properties, such as one or more of the medical device(s) disclosed in WIPO Publication No. WO 2013/192574 A1 to Faulkner et al., published Dec. 27, 2013, PCT Application No. PCT/US2013/047159 to Faulkner et al., filed Jun. 21, 2013, United States Patent Application Publication No. US 2007/0112333 to Hoang et al., published May 17, 2007; U.S. Pat. No. 7,780,794 to Rogers et al., issued Aug. 24, 2010; United States Patent Application Publication No. US 2011/0044850 to Solomon et al., published Feb. 24, 2011; United States Patent Application Publication No. US 2011/0217212 to Solomon et al., published Sep. 8, 2011; and United States Patent Application Publication No. US 2011/0232020 to Rogers et al., published Sep. 29, 2011, the disclosure of each of which is hereby incorporated herein in its entirety by this reference.

There remains a need for new structures, devices, assemblies, and methods facilitating the simple and efficient cleaning of potentially contaminated surfaces of a medical device.

### BRIEF SUMMARY

Described are antimicrobial devices, assemblies including antimicrobial devices, and methods of decontaminating a medical device. For example, in accordance with one embodiment described herein, an antimicrobial device comprises a housing structure configured to couple to a medical device and comprising an antimicrobial polymer material comprising polyurethane. The antimicrobial polymer material is formulated to at least partially decontaminate surfaces of the medical device in at least temporary physical contact with the housing structure.

In additional embodiments, a method of decontaminating a medical device comprises coupling the medical device to an antimicrobial device comprising an antimicrobial polymer material comprising polyurethane, the antimicrobial polymer material formulated to at least partially decontaminate surfaces of the medical device in at least temporary physical contact with surfaces of the antimicrobial device.

In further embodiments, a medical device assembly comprises a medical device, and an antimicrobial device coupled to the medical device and comprising an antimicrobial polymer material comprising polyurethane. The antimicrobial polymer material is formulated to at least partially sterilize at least one surface of the medical device in physical contact with at least one surface of the antimicrobial device.

### BRIEF DESCRIPTION OF THE DRAWINGS

FIG. 1 illustrates a side elevation view of an antimicrobial device, in accordance with an embodiment of the disclosure.

FIG. 2 illustrates a cross-sectional side view of the antimicrobial device shown in FIG. 1.

FIG. 3 illustrates a cross-sectional side view of a medical device assembly including the antimicrobial device shown in FIGS. 1 and 2.

FIG. 4 illustrates a perspective view of an antimicrobial device, in accordance with another embodiment of the disclosure.

FIG. 5 illustrates a cross-sectional side view of the antimicrobial device shown in FIG. 4.

FIG. 6 illustrates a cross-sectional side view of a medical device assembly including the antimicrobial device shown in FIGS. 4 and 5.

FIG. 7 illustrates a perspective view of an antimicrobial device, in accordance with an additional embodiment of the disclosure.

FIG. 8 illustrates a cross-sectional side view of the antimicrobial device shown in FIG. 7.

FIG. 9 illustrates a cross-sectional side view of a medical device assembly including the antimicrobial device shown in FIGS. 7 and 8.

FIG. 10 illustrates a cross-sectional side view of another medical device assembly including the antimicrobial device shown in FIGS. 7 and 8.

### DETAILED DESCRIPTION

Antimicrobial devices are disclosed, as are medical device assemblies including antimicrobial devices, and methods of decontaminating a medical device. In some embodiments, an antimicrobial device includes a housing structure configured to couple (e.g., connect, attach, etc.) to a medical device. The housing structure may at least partially include (e.g., may be at least partially formed of and include) an antimicrobial polymer material comprising an antimicrobial elastomer, such as polyurethane. The antimicrobial polymer material may be formulated to at least partially (e.g., substantially) decontaminate (e.g., disinfect, sterilize, clean, etc.) surfaces of the medical device in at least temporary physical contact with the housing structure. During use and operation, the antimicrobial device may be coupled to the medical device for a predetermined amount of time to substantially destroy and prevent the propagation of one or more pathogens that may be present on at least a portion of the medical device, and/or to substantially protect the portion of the medical device from future pathogenic contamination. The antimicrobial devices, medical device assemblies, and methods of the disclosure may provide enhanced safety, improved simplicity, and/or reduced costs relative to many conventional antimicrobial devices, medical device assemblies, and methods associated with medical treatment applications.

The following description provides specific details, such as material types, shapes, sizes, and processing conditions in order to provide a thorough description of embodiments of the disclosure. However, a person of ordinary skill in the art will understand that the embodiments of the disclosure may be practiced without employing these specific details. Indeed, the embodiments of the disclosure may be practiced in conjunction with conventional fabrication techniques employed in the industry. In addition, the description provided below does not form a complete process flow for manufacturing a structure, device, or assembly. The structures described below do not necessarily faun a complete device or a complete assembly. Only those process acts and structures necessary to understand the embodiments of the disclosure are described in detail below. Additional acts to form a complete device or a complete assembly from various structures described herein may be performed by conventional fabrication processes.

Drawings presented herein are for illustrative purposes only, and are not meant to be actual views of any particular material, component, structure, device, or assembly. Variations from the shapes depicted in the drawings as a result, for example, of manufacturing processes and/or tolerances, are to be expected. Thus, embodiments described herein are not to be construed as being limited to the particular shapes or regions as illustrated, but include deviations in shapes that result, for example, from manufacturing. For example, a region illustrated or described as box-shaped may have rough and/or nonlinear features, and a region illustrated or described as round may include some rough and/or linear features. Moreover, sharp angles that are illustrated may be rounded, and vice versa. Thus, the regions illustrated in the figures are schematic in nature, and their shapes are not intended to illustrate the precise shape of a region and do not limit the scope of the present claims. The drawings are not necessarily to scale. Additionally, elements common between figures may retain the same numerical designation.

As used herein, the terms “comprising,” “including,” “containing,” “characterized by,” and grammatical equivalents thereof are inclusive or open-ended terms that do not exclude additional, unrecited elements or method acts, but also include the more restrictive terms “consisting of” and “consisting essentially of” and grammatical equivalents thereof. As used herein, the term “may” with respect to a material, structure, feature or method act indicates that such is contemplated for use in implementation of an embodiment of the disclosure and such term is used in preference to the more restrictive term “is” so as to avoid any implication that other, compatible materials, structures, features and methods usable in combination therewith should or must be, excluded.

As used herein, the singular forms “a,” “an,” and “the” are intended to include the plural forms as well, unless the context clearly indicates otherwise.

As used herein, the term “and/or” includes any and all combinations of one or more of the associated listed items.

As used herein, spatially relative terms, such as “beneath,” “below,” “lower,” “bottom,” “above,” “upper,” “top,” “front,” “rear,” “left,” “right,” and the like, may be used for ease of description to describe one element's or feature's relationship to another element(s) or feature(s) as illustrated in the figures. Unless otherwise specified, the spatially relative terms are intended to encompass different orientations of the materials in addition to the orientation depicted in the figures. For example, if materials in the figures are inverted, elements described as “below” or “beneath” or “under” or “on bottom of” other elements or features would then be oriented “above” or “on top of” the other elements or features. Thus, the term “below” can encompass both an orientation of above and below, depending on the context in which the term is used, which will be evident to one of ordinary skill in the art. The materials may be otherwise oriented (e.g., rotated 90 degrees, inverted, flipped, etc.) and the spatially relative descriptors used herein interpreted accordingly.

As used herein, the term “substantially” in reference to a given parameter, property, or condition means and includes to a degree that one of ordinary skill in the art would understand that the given parameter, property, or condition is met with a degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” in reference to a given parameter is inclusive of the stated value and has the meaning dictated by the context (e.g., it includes the degree of error associated with measurement of the given parameter).

As used herein, the term “configured” refers to a size, shape, material composition, and arrangement of one or more of at least one structure and at least one apparatus facilitating operation of one or more of the structure and the apparatus in a predetermined way.

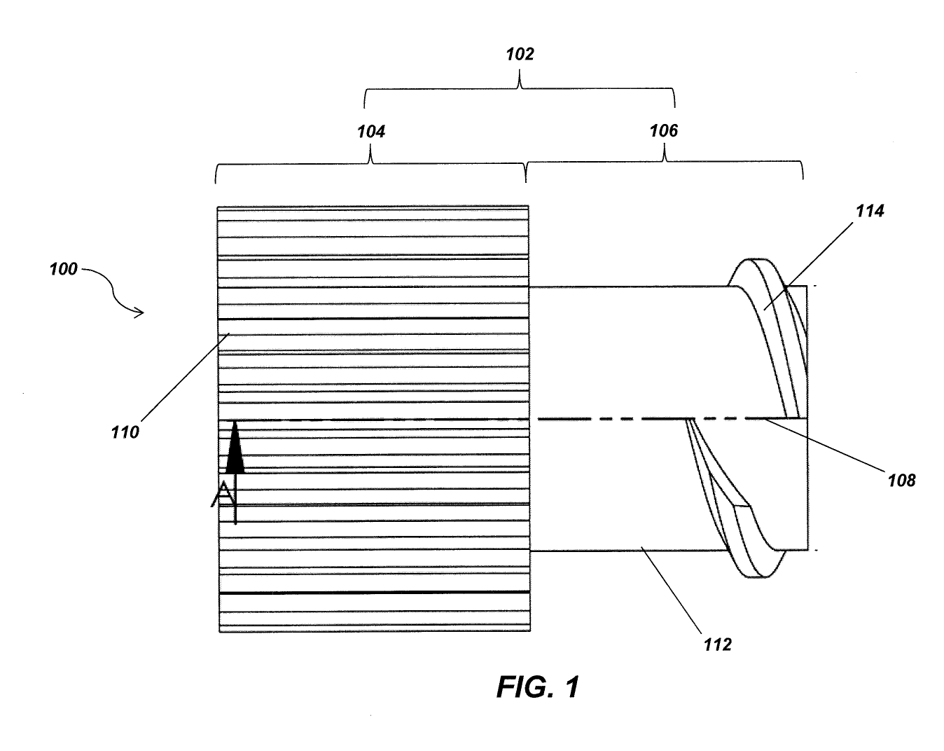

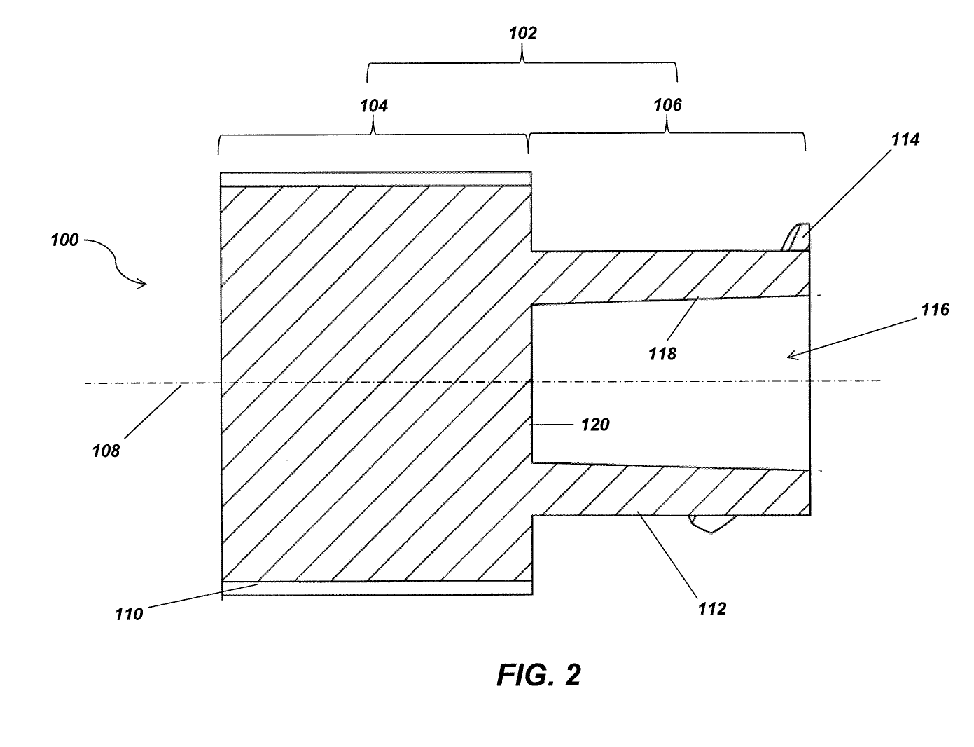

FIG. 1 illustrates a side elevation view of an antimicrobial device 100 (e.g., an antimicrobial cap, such as an antimicrobial medical device cleaning cap), in accordance with an embodiment of the disclosure. As shown in FIG. 1, the antimicrobial device 100 includes a housing structure 102 exhibiting a head region 104 and a stem region 106. The antimicrobial device 100 is configured to attach to and at least partially (e.g., substantially) decontaminate (e.g., disinfect, sterilize, clean, etc.) at least a portion of a medical device, as described in further detail below. FIG. 2 is a cross-sectional side view of the antimicrobial device 100 depicted in FIG. 1. While FIGS. 1 and 2 depict a particular configuration of the antimicrobial device 100, one of ordinary skill in the art will appreciate that the antimicrobial device 100 may exhibit a different configuration, such as a configuration exhibiting at least one of a different size, a different shape, different features, and different feature spacing. FIGS. 1 and 2 illustrate just one non-limiting example of the antimicrobial device 100.

As shown in FIGS. 1 and 2, the head region 104 may be integral and continuous with the stem region 106, and may extend laterally outward beyond a periphery of the stem region 106. As used herein with respect to the antimicrobial device 100, each of the terms “lateral” and “horizontal” means and includes extending in a direction substantially perpendicular (e.g., orthogonal) to a central axis 108 of the antimicrobial device 100, regardless of the orientation of the antimicrobial device 100. Accordingly, as used herein with respect to the antimicrobial device 100, each of the terms “longitudinal” and “vertical” means and includes extending in a direction substantially parallel to the central axis 108 of the antimicrobial device 100, regardless of the orientation of the antimicrobial device 100. The head region 104 may exhibit any desired dimensions (e.g. shape and size). In some embodiments, the head region 104 exhibits a generally cylindrical shape. Furthermore, as depicted in FIGS. 1 and 2, the head region 104 may, optionally, exhibit one or more ribs 110 extending (e.g., longitudinally extending) along a periphery thereof The ribs 110, if present, may assist a user (e.g., a practitioner, a healthcare provider, etc.) in gripping and manipulating the antimicrobial device 100 during use and operation.

The stem region 106, which may also be identified as a connection region (e.g., a coupling region, an attachment region, etc.), longitudinally projects from the head region 104, and is configured for at least temporary attachment with at least one connection structure and/or at least one connection region of a medical device. The stem region 106 may exhibit any desired dimensions (e.g., shape and size) facilitating attachment to the connection structure and/or the connection region of the medical device. In some embodiments, the stem region 106 exhibits a generally tubular shape. As shown in FIGS. 1 and 2, at least a portion of an outer surface 112 of the stem region 106 may, optionally, exhibit threads 114 for coupling with a female connection structure (e.g., a female Luer connection) of the medical device. In additional embodiments, the threads 114 may be omitted (e.g., absent) from the stem region 106. In such embodiments, the stem region 106 may, optionally, include different means (e.g., one or more flexible protrusions, interference fit, surface friction between components, etc.) for coupling with a female connection region of a given medical device.

As shown in FIG. 2, an opening 116 (e.g., bore, via, recess, cavity, etc.) at least partially extends through the antimicrobial device 100. In some embodiments, the opening 116 comprises a blind opening that extends at least through the stem region 106 of the housing structure 102 of the antimicrobial device 100. By way of non-limiting example, the opening 116 may extend partially through and terminate within the stem region 106, may extend completely through the stem region 106 and terminate at the head region 104, or may extend completely through the stem region 106 and partially into the head region 104. In additional embodiments, the opening 116 comprises a through opening that extends completely through each of the stem region 106 and the head region 104 of the housing structure 102 of the antimicrobial device 100. The opening 116 may be configured (e.g., shaped and sized) to at least partially receive a male connection structure (e.g., a male Luer connection) of a medical device to facilitate the at least temporary retention and at least partial (e.g., substantial) decontamination (e.g., sterilization, disinfection, etc.) of the male connection structure, as described in further detail below. For example, the opening 116 may be configured such that at least one of inner sidewalls 118 and a floor 120 (if present) of the housing structure 102 defining the opening 116 abut against (e.g., directly physically contact) one or more portion(s) of the male connection structure.

In some embodiments, the housing structure 102 of the antimicrobial device 100 is configured to maximize the amount of contact between surfaces of the housing structure 102 and surfaces of a medical device connected thereto and/or received therein. The housing structure 102 of the antimicrobial device 100 may, for example, be configured to maximize the amount of surface area of the housing structure 102 that directly physically contacts the surface area of the medical device. By way of non-limiting example, the stem region 106 of the housing structure 102 of the antimicrobial device 100 may be configured to maximize contact between the stem region 106 and a corresponding structure of the medical device.

The antimicrobial device 100 may be formed of and include at least one antimicrobial polymer material exhibiting antimicrobial properties sufficient to substantially decontaminate at least a portion (e.g., a connection portion) of a medical device that may come in physical contact therewith. The antimicrobial polymer material may, for example, be formulated to substantially decontaminate surfaces (e.g., surfaces of a connection structure) of the medical device in physical contact with one or more surface(s) (e.g., at least one of the outer surface 112 of the stem region 106, the inner sidewalls 118 of the housing structure 102 at least partially defining the opening 116, the floor 120 of the housing structure 102 at least partially defining the opening 116, etc.) of the antimicrobial device 100. The antimicrobial polymer material may destroy (e.g., kill) and/or prevent the propagation (e.g., growth, multiplication, etc.) of one or more (e.g., substantially all) infection causing pathogen(s) (e.g., microorganisms, bacteria, undulating bacteria, spirochetes, spores, spore-forming organisms, gram-negative organisms, gram-positive organisms, yeasts, fungi, molds, viruses, aerobic organisms, anaerobic organisms, mycobacteria, etc.) on the surface(s) of the medical device in physical contact with the surface(s) of the antimicrobial device 100. The antimicrobial polymer material may be formulated to substantially decontaminate the surfaces of the medical device of one or more infection causing pathogen(s) substantially immediately (e.g., upon physical contact with the surface of the antimicrobial device 100) and/or gradually (e.g., over the course of prolonged physical contact with the surface of the antimicrobial device 100).

Examples of infection causing pathogens that may be destroyed through physical contact (e.g., substantially immediately and/or gradually) with the antimicrobial polymer material of the antimicrobial device 100 include, but are not limited to, Acinetobacter baumannii, Actinobacter spp, Aspergillus niger, Aspergillus flavus, Aureobasidium pullulans, Bacillus cereus, Bacillus thuringiensis, Bovine Viral Diarrhea Virus (Surrogate for Human Hepatitis C Virus), Candida albicans, Chaetomium globosum, Citrobacter diversus, Cladosporium herbarium, Clostridium diffacile, Enterobacter aerogines, Enterobacter cloacae, Enterobacteriaceae, Carbapenem-resistant Enterobacteriaceae (CRE), Enterococcus, Source and Rhinovirus and Vancomycin-resistant Enterococcus (VRE), Enterococcus faecalis, Epidennophyton floccosum, Escherichia coli, Gliocladtum vixens, Histoplasma capsulatum, Klebsiella oxytoca, Klebsiella pneumoniae, Legionella pneumpophila, Listeria monocytogenes, Mycobacterium tuberculosis, Penicillium fimiculosum, Porphyromonas gingivalis, Proteus mirabilis, Proteus vulgaris, Pseudomonas aeruginosa, Rhizopus nigricans, S. saprophyticus, Saccharomyces cerevisiae, Salmonella gallinarum, Salmonella typhimurium, Stachybotrys, Staphylococcus aureus, Methicillin-resistant Staphylococcus aureus (MRSA), Staphylococcus epidermidis, Streptococcus agalactiae, Streptococcus faecalis, Streptococcus pneumoniae, Streptococcus mutans, Trichophyton mentagrophytes, Trycophyton malmsten, and/or Vibrio parahaemolyticus.

By way of non-limiting example, the antimicrobial polymer material of the antimicrobial device 100 may be formed of and include at least one antimicrobial elastomer, and, optionally, at least one antimicrobial additive. As used herein, the term “antimicrobial elastomer” means and includes an elastomeric compound exhibiting antimicrobial (e.g., biocidal) properties. The antimicrobial elastomer may comprise a solid, at least partially cross-linked polymer exhibiting rubbery elastic extensibility and restoring properties and antimicrobial properties. The antimicrobial elastomer may impart the antimicrobial device 100 with one or more biocidal, contact-active surfaces. For example, the antimicrobial elastomer may exhibit biocidal end groups permitting surfaces of the antimicrobial device 100 to destroy and/or prevent the propagation of pathogens on the surface of a medical device upon contact. In some embodiments, the antimicrobial device 100 is formed of and includes polyurethane, such as thermoplastic polyurethane (TPU). As used herein, the term “polyurethane” means and includes an elastomeric polymer produced through a condensation reaction between a polyisocyanate and a hydroxyl-containing material. Suitable polyurethanes include, but are not limited to, polyether-based polyurethanes, polyester-based polyurethanes, poly(ether urethane urea), silicone urethanes (e.g., aliphatic diisocyanates in combination with polyether, aromatic polyester, and/or aliphatic polyester; aromatic diisocyanates in combination with polyether, aromatic polyester, and/or aliphatic polyester; etc.). For example, the antimicrobial elastomer may comprise a polyurethane exhibiting antimicrobial moieties, such as one or more of the polyurethanes described in U.S. Patent Application Publication No. 2011/0195041 to Chisholm et. al, the entire disclosure of which is hereby incorporated herein by reference in its entirety. In some embodiments, the antimicrobial elastomer (e.g., polyurethane) is sufficient, by itself (e.g., without the presence of an antimicrobial additive), to substantially decontaminate at least one surface of a medical device in physical contact with at least one surface of the antimicrobial device 100 of one or more (e.g., substantially all) infection causing pathogen(s). In additional embodiments, the antimicrobial elastomer (e.g., polyurethane), in combination with the antimicrobial additive, is sufficient to substantially decontaminate at least one surface of a medical device in physical contact with at least one surface of the antimicrobial device 100 of one or more (e.g., substantially all) infection causing pathogen(s).

Although embodiments of the disclosure are discussed below with reference to the use of an antimicrobial polymer material formed of and including polyurethane, in other embodiments, the antimicrobial polymer material may be formed of and include a different antimicrobial elastomer, such as one or more of butyl rubber, ethylene propylene diene monomer (EPDM) rubber, silicone rubber, and/or polyisoprene. Initial testing involving the decontamination of medical devices has indicated that antimicrobial polymer materials comprising polyurethane may produce substantial decontamination results for a majority of medical device applications.

If present, the antimicrobial additive may comprise at least one material that is compatible with the antimicrobial elastomer (e.g., silicone rubber) and that promotes the antimicrobial properties of the antimicrobial device 100. As used herein, the term “compatible” means that a material does not undesirably react, decompose, or absorb another material, and also that the material does not undesirably impair the chemical and/or mechanical properties of the another material. Non-limiting examples of suitable antimicrobial additives include silver-based antimicrobial agents (e.g., silver, silver nitrate, silver citrate, silver sulfadiazine, silver acetate, silver sulphate, silver chloride, silver oxide, silver-silica composites, chlorhexidine-silver sulfadiaxine, sodium silver hydrogen zirconium phosphate, Agion, Triclosan, etc.), non-silver-based antimicrobial agents (e.g., alcohols; boron; biguanide polymers; chlorhexidine; chlorhexidine gluconate; chlorhexidine diacetate; chlorhexidine dihydrate; active chlorine; copper; iodine; phenolic-based materials, such as phenol, 5-chloro-2-(2,4-dichlorpheynoyl(phenol), etc.; quaternary ammonium-based materials, such as benzalkonium chloride; zinc-based materials, such as zinc, zinc pyrithione, zinc omadine, etc.; etc.), or combinations thereof. The type and amount of the antimicrobial additive present (if any) within the antimicrobial polymer material may selected relative to the type and amount of antimicrobial elastomer to impart the antimicrobial device 100 with desired antimicrobial properties (e.g., biocidal effects against one or more infection causing pathogen(s), a desired decontamination rate against a surface of a medical device in contact with the antimicrobial device 100, etc.). By way of non-limiting example, the antimicrobial polymer material may include from about 0 percent by weight (wt %) to about 20 wt % of the antimicrobial additive, such as from about 0.1 wt % to about 10 wt % of the antimicrobial additive, or from about 0.5 wt % to about 5 wt % of the antimicrobial additive.

Suitable antimicrobial polymer materials (e.g., including an antimicrobial elastomer, such as a polyurethane; and, optionally, at least one antimicrobial additive, such as a silver-based antimicrobial agent and/or a non-silver-based antimicrobial agent) are commercially available from, for example, Parker Hannifin Corporation (Salt Lake City, Utah). In some embodiments, the antimicrobial polymer material of the antimicrobial device 100 is formed of and includes polyurethane and at least one antimicrobial additive, such as one or more of the antimicrobial polymer materials described in at least one of U.S. Pat. No. 6,846,871 to Patel et al., U.S. Pat. No. 7,772,296 to Garey et al., and U.S. Patent Publication No. 2010/0240800 to Cunningham, the disclosure of each of which is hereby incorporated herein in its entirety by this reference.

The antimicrobial device 100 may be substantially homogeneous (e.g., the antimicrobial device 100 may consist of the antimicrobial elastomer, or the antimicrobial elastomer and the antimicrobial additive may be substantially uniformly distributed throughout the antimicrobial device 100), or may be heterogeneous (e.g., the antimicrobial elastomer and/or the antimicrobial additive may be non-uniformly distributed throughout the antimicrobial device 100). In some embodiments, the antimicrobial device 100 is substantially homogeneous, and is substantially free of antimicrobial additive(s) (e.g., the antimicrobial device 100 is formed of and includes at least one antimicrobial elastomer, such as polyurethane, but not at least one antimicrobial additive). In additional embodiments, the antimicrobial device 100 is substantially homogeneous, and is formed of and includes at least one antimicrobial elastomer (e.g., polyurethane) and at least one antimicrobial additive. In further embodiments, the antimicrobial device 100 is substantially heterogeneous, and is formed of and includes at least one antimicrobial elastomer (e.g., polyurethane) and at least one antimicrobial additive. In embodiments wherein the antimicrobial device 100 is substantially heterogeneous, the antimicrobial device 100 may, for example, exhibit a linear concentration distribution of the antimicrobial additive, a stepwise concentration distribution of the antimicrobial additive, or a Gaussian concentration distribution of the antimicrobial additive throughout a thickness of the antimicrobial device 100. By way of non-limiting example, the antimicrobial device 100 may exhibit a relatively higher concentration of the antimicrobial additive proximate one or more surface(s) (e.g., at least one of the outer surface 112 of the stem region 106, the inner sidewalls 118 of the housing structure 102 at least partially defining the opening 116, the floor 120 of the housing structure 102 at least partially defining the opening 116, etc.) of the antimicrobial device 100 as compared to other portions of the antimicrobial device 100.

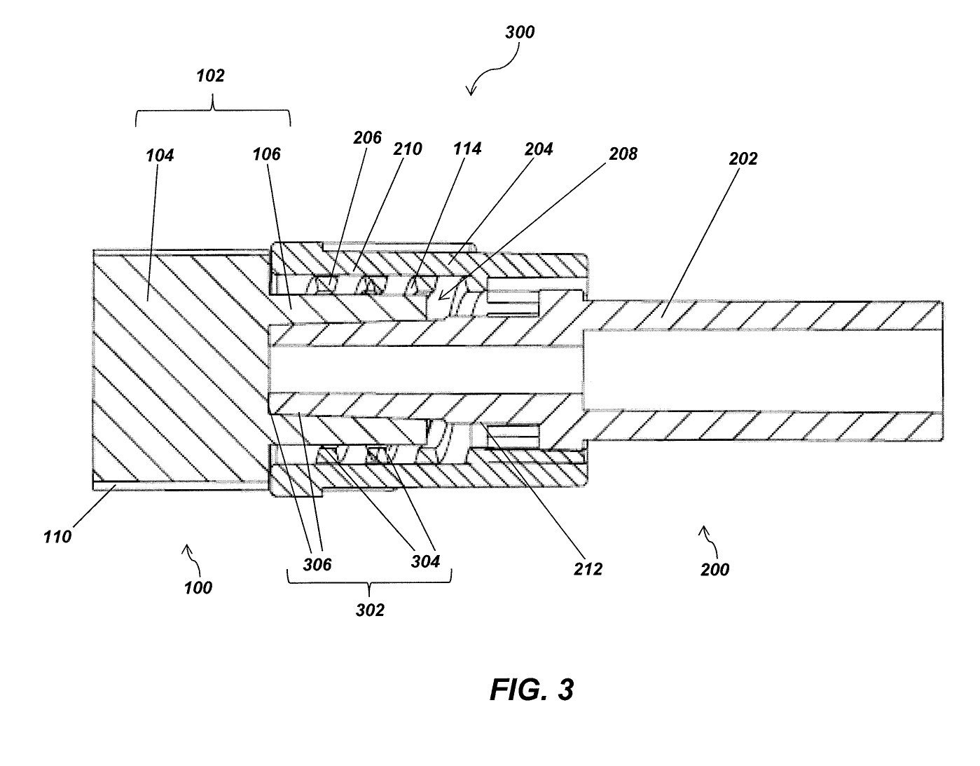

FIG. 3 is a cross-sectional side view of a medical device assembly 300, in accordance with an embodiment of the disclosure. The medical device assembly 300 may include a medical device 200, and the antimicrobial device 100 previously described in relation to FIGS. 1 and 2. The antimicrobial device 100 may be coupled to the medical device 200. As shown in FIG. 3, the medical device 200 may include a male connection structure 202 (e.g., a male luer structure) and a hub structure 204 movably connected to and at least partially surrounding the male connection structure 202. The male connection structure 202 and the hub structure 204 may be configured (e.g., sized and shaped) relative to one another such that the hub structure 204 may slide along and rotate about the male connection structure 202. In addition, at least the stem region 106 of the housing structure 102 of the antimicrobial device 100 may be configured to at least partially receive and retain the male connection structure 202 of the medical device 200 within the opening 116 (FIG. 2) at least partially extending through the antimicrobial device 100, and may also be configured to be at least partially received and retained within another opening 208 of the medical device 200 at least partially defined by opposing sidewalls of the male connection structure 202 and the hub structure 204. As depicted in FIG. 3, the threads 114 projecting from the outer surface 112 (FIG. 2) of the stem region 106 of the antimicrobial device 100 may be configured (e.g., sized, shaped, spaced, etc.) to couple with additional threads 206 projecting from an inner surface 210 of the hub structure 204 of the medical device 200. While FIG. 3 depicts a particular configuration of the medical device assembly 300, one of ordinary skill in the art will appreciate that different medical device assembly configurations are known in the art, which may be adapted to be employed in embodiments of the disclosure. FIG. 3 illustrates just one non-limiting example of the medical device assembly 300. For example, in additional embodiments, the antimicrobial device 100 may include structures other than the threads 114 for coupling with the medical device 200, such as at least one flexible protrusion that engages with the threads 206 of the medical device 200 and also maximizes surface contact between the outer surface of the stem 106 of the antimicrobial device 100 and the inner surface of the hub structure 204 of the medical device 200.

As shown in FIG. 3, surfaces of the antimicrobial device 100 may at least temporarily directly physically contact (e.g., abut) surfaces of the medical device 200 at interfaces 302. As a non-limiting example, the outer surface 112 (FIG. 2) of the stem region 106 of the antimicrobial device 100 may temporarily directly physically contact the inner surface 210 of the hub structure 204 at first interfaces 304. As another non-limiting example, at least one of the inner sidewalls 118 (FIG. 2) and the floor 120 (FIG. 2) of the housing structure 102 defining the opening 116 (FIG. 2) within the antimicrobial device 100 may temporarily directly physically contact an outer surface 212 of the male connection structure 202 at second interfaces 306. The antimicrobial device 100 may substantially decontaminate the portions of the medical device 200 in direct physical contact therewith at the interfaces 302.

One or more portion(s) of the medical device 200 (e.g., surface(s) of the male connection structure 202, surface(s) of the hub structure 204, etc.) may be provided in direct physical contact with the antimicrobial device 100 for any amount of time (e.g., duration) sufficient to substantially decontaminate the portion(s) of the medical device 200 and/or to substantially protect the portion(s) of the medical device 200 from future contamination. In some embodiments, the antimicrobial device 100 remains coupled to and in direct physical contact the medical device 200 for a period of time less than or equal to about 24 hours, such as less than or equal to about 12 hours, less than or equal to about 6 hours, less than or equal to about 3 hours, less than or equal to about 1 hour, less than or equal to about 30 minutes, less than or equal to about 15 minutes, less than or equal to about 5 minutes, less than or equal to about 1 minute, or less than or equal to about 30 seconds. For example, one or more portion(s) of the medical device 200 may be brought into and maintained in direct physical contact with one or more portion(s) of the antimicrobial device 100 for a brief period of time less than or equal to about 1 minute to substantially decontaminate the portion(s) of medical device 200 directly (e.g., immediately) prior to a desired application (e.g., a medical treatment application, such as material injection application) of the medical device 200. in additional embodiments, the antimicrobial device 100 remains coupled to and in direct physical contact with the medical device 200 for a period of time greater than about 24 hours, such as greater than or equal to about three (3) days, greater than or about to about one (1) week, greater than or about to about two (2) weeks, greater than or about to about one (1) month, greater than or about to about three (3) months, or greater than or about to about six (6) months. For example, one or more portion(s) of the medical device 200 may be brought into and maintained in direct physical contact with one or more portion(s) of the antimicrobial device 100 for a prolonged period of time greater than or equal to about 24 hours to ensure the medical device 200 is and remains substantially decontaminated (e.g., substantially free of pathogens) until a desired application. Prior to bringing the medical device 200 into direct physical contact (e.g., brief direct physical contact, prolonged physical contact, etc.) with the antimicrobial device 100, the medical device 200 may have been used for another desired application (e.g., another medical treatment application, such as another material injection application), or the desired application may be the first use of the medical device 200.

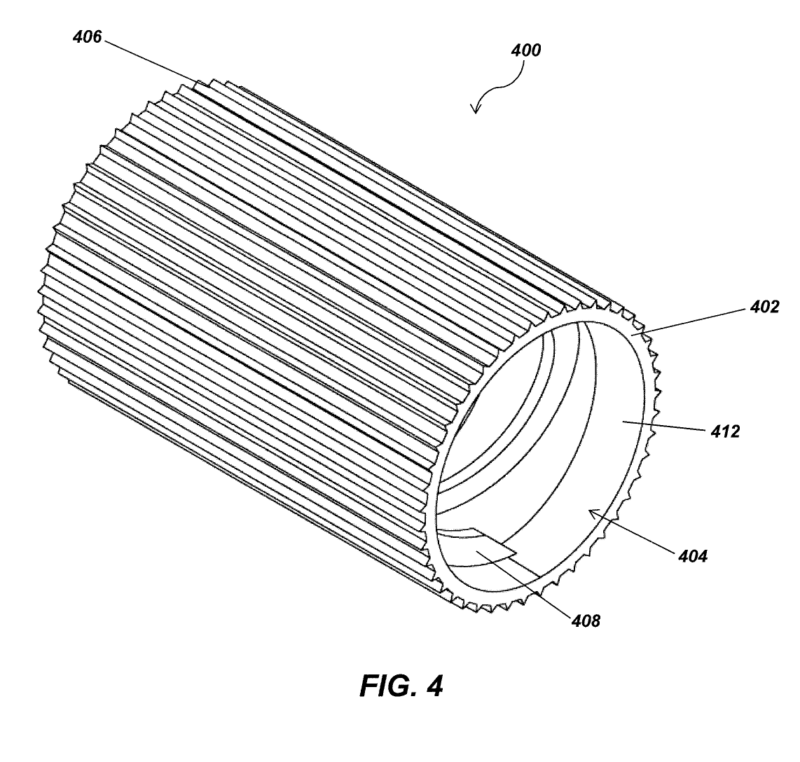

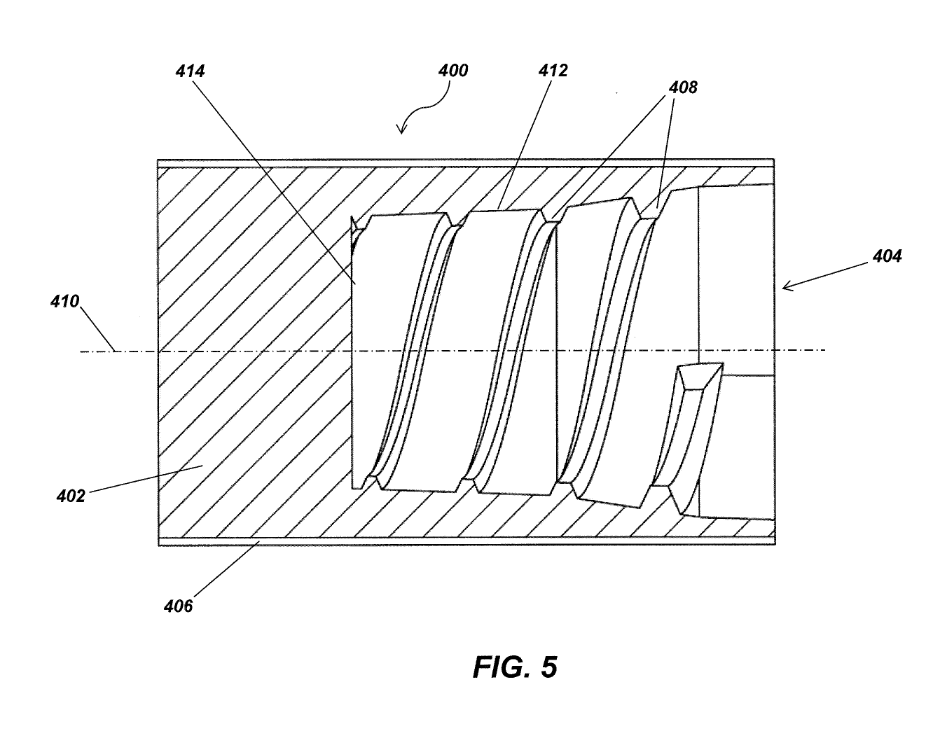

FIG. 4 illustrates a perspective view of an antimicrobial device 400 (e.g., an antimicrobial cap, such as an antimicrobial medical device cleaning cap), in accordance with another embodiment of the disclosure. The antimicrobial device 400 is configured to attach to and at least partially (e.g., substantially) decontaminate (e.g., disinfect, sterilize, clean, etc.) at least a portion of a medical device, as described in further detail below. FIG. 5 is a cross-sectional side view of the antimicrobial device 400 depicted in FIG. 4. While FIGS. 4 and 5 depict a particular configuration of the antimicrobial device 400, one of ordinary skill in the art will appreciate that the antimicrobial device 400 may exhibit a different configuration, such as a configuration exhibiting at least one of a different size, a different shape, different features, and different feature spacing. FIGS. 4 and 5 illustrate just one non-limiting example of the antimicrobial device 400.

As shown in FIGS. 4 and 5, the antimicrobial device 400 may include a housing structure 402 exhibiting an opening 404 (e.g., bore, via, recess, cavity, etc.) at least partially longitudinally extending therethrough. As used herein with respect to the antimicrobial device 400, each of the terms “longitudinal” and “vertical” means and includes extending in a direction substantially parallel to a central axis 410 (FIG. 5) of the antimicrobial device 400, regardless of the orientation of the antimicrobial device 400. Accordingly, as used herein with respect to the antimicrobial device 400, each of the terms “lateral” and “horizontal” means and includes extending in a direction substantially perpendicular (e.g., orthogonal) to the central axis 410 of the antimicrobial device 400, regardless of the orientation of the antimicrobial device 400. The housing structure 402 may exhibit any desired dimensions (e.g. shape and size). In some embodiments, the housing structure 402 exhibits a generally cylindrical shape. Furthermore, as depicted in FIGS. 4 and 5, the housing structure 402 may, optionally, exhibit one or more ribs 406 extending (e.g., longitudinally extending) along a periphery thereof. The ribs 406, if present, may assist a user (e.g., a practitioner, a healthcare provider, etc.) in gripping and manipulating the antimicrobial device 400 during use and operation.

As shown in FIG. 5, in some embodiments, the opening 404 comprises a blind opening extending partially through and terminating within the housing structure 402. In additional embodiments, the opening 404 comprises a through opening extending completely through the housing structure 402. The opening 404 may be configured (e.g., shaped and sized) to at least partially receive a male connection structure (e.g., a male Luer connection) of a medical device to facilitate the at least temporary retention and at least partial (e.g., substantial) decontamination (e.g., sterilization, disinfection, etc.) of the male connection structure, as described in further detail below. The opening 404 may, for example, be configured such that at least one of inner sidewalls 412 and a floor 414 (if present) of the housing structure 402 defining the opening 404 abut against (e.g., directly physically contact) portions of the male connection structure. In some embodiments, the opening 404 exhibits a tapered shape (e.g., a frustoconical shape) wherein one end (e.g., the end proximate the floor 414) of the opening 404 has a relatively smaller area and/or a relatively smaller volume than another end (e.g., the end distal from the floor 414) of the opening 404. In additional embodiments, the opening 404 may exhibit a different shape, such as a substantially non-tapered shape. As shown in FIGS. 4 and 5, at least a portion of the inner sidewalls 412 of the housing structure 402 may, optionally, exhibit threads 408 for coupling with a male connection structure (e.g., a male Luer connection) of a medical device. In such embodiments, the inner sidewalls 412 may, optionally, exhibit different means (e.g., one or more flexible protrusions, interference fit, surface friction between components, etc.) for coupling with a connection structure of a given medical device (e.g., in a manner similar to that discussed above in relation to the antimicrobial device 100 to maximize contact between the antimicrobial device 400 and the medical device).

In some embodiments, the housing structure 402 of the antimicrobial device 400 is configured to maximize the amount of contact between surfaces of the housing structure 402 and surfaces of a medical device connected thereto and/or received therein. in a manner similar to that previously discussed in relation to antimicrobial device 100.

The material composition of the antimicrobial device 400 may be substantially similar to the material composition of the antimicrobial device 100 previously described with respect to FIGS. 1 and 2. For example, antimicrobial device 400 may be formed of and include at least one antimicrobial polymer material exhibiting antimicrobial properties sufficient to substantially disinfect (e.g., substantially sterilize, substantially decontaminate, etc.) at least a portion of a medical device in at least temporary physical contact with the antimicrobial device 400. The antimicrobial polymer material may be formed of and include at least one antimicrobial elastomer (e.g., polyurethane), and, optionally, at least one antimicrobial additive (e.g., a silver-based antimicrobial agent, a non-silver-based antimicrobial agent, combinations thereof, etc.). In some embodiments, the antimicrobial device 400 is formed of and includes polyurethane. In addition, the antimicrobial device 400 may be substantially homogeneous (e.g., the antimicrobial device 400 may consist of the antimicrobial elastomer, or the antimicrobial elastomer and the antimicrobial additive may be substantially uniformly distributed throughout the antimicrobial device 400), or may be heterogeneous (e.g., the antimicrobial elastomer and/or the antimicrobial additive may be non-uniformly distributed throughout the antimicrobial device 400). In some embodiments, the antimicrobial device 400 is substantially homogeneous, and is substantially free of antimicrobial additive(s) (e.g., the antimicrobial device 400 is formed of and includes at least one antimicrobial elastomer, such as polyurethane, but not at least one antimicrobial additive). In additional embodiments, the antimicrobial device 400 is substantially homogeneous, and is formed of and includes at least one antimicrobial elastomer (e.g., polyurethane) and at least one antimicrobial additive. In further embodiments, the antimicrobial device 400 is substantially heterogeneous, and is formed of and includes at least one antimicrobial elastomer (e.g., polyurethane) and at least one antimicrobial additive.

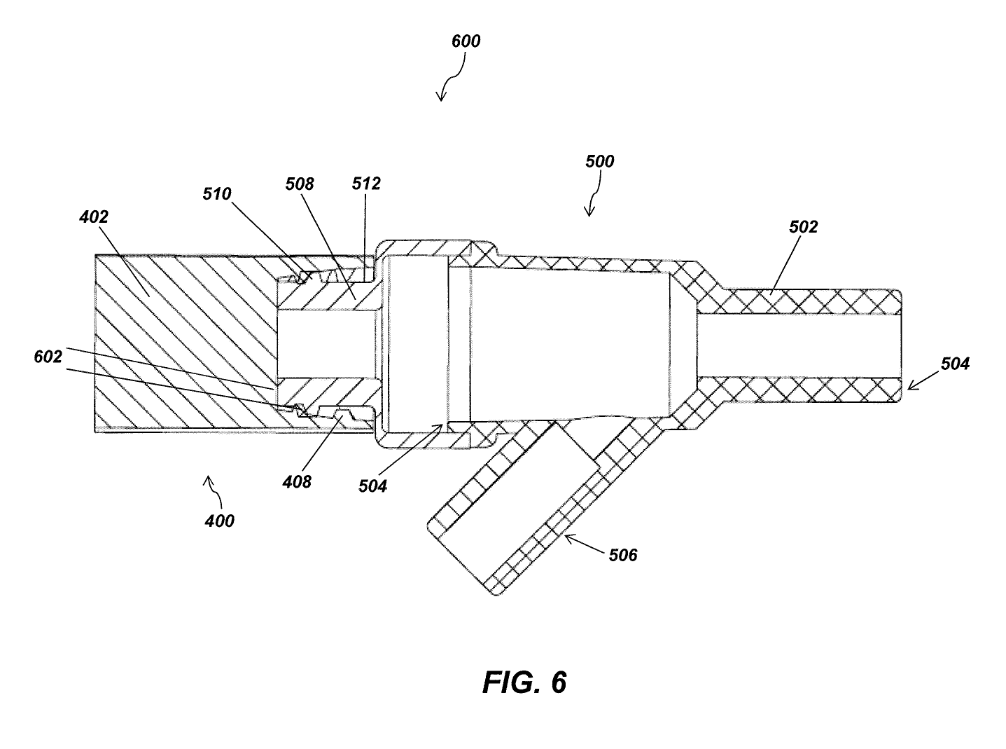

FIG. 6 is a cross-sectional side view of a medical device assembly 600, in accordance with an embodiment of the disclosure. The medical device assembly 600 includes a medical device 500 (e.g., needleless luer access port, Y-connector, etc.), and the antimicrobial device 400 previously described in relation to FIGS. 4 and 5. The antimicrobial device 400 may be coupled to the medical device 500. As shown in FIG. 6, the medical device 500 may include a housing structure 502 exhibiting opposing ends 504 and a branched region 506, and a male connection structure 508 coupled to (e.g., attached to. bonded to, welded to, etc.) one of the opposing ends 504 of the housing structure 502. The housing structure 402 of the antimicrobial device 400 may be configured to receive and retain at least a portion of the male connection structure 508 of the medical device 500 within the opening 404 (FIGS. 4 and 5) at least partially extending through the antimicrobial device 400. As depicted in FIG. 6, the threads 408 projecting from the inner sidewalls 412 (FIGS. 4 and 5) of the housing structure 402 of the antimicrobial device 400 may be configured (e.g., sized, shaped, spaced, etc.) to couple with additional threads 510 extending from an outer surface 512 of the male connection structure 508 of the medical device 500. While FIG. 6 depicts a particular configuration of the medical device assembly 600, one of ordinary skill in the art will appreciate that different medical device assembly configurations are known in the art, which may be adapted to be employed in embodiments of the disclosure. FIG. 6 illustrates just one non-limiting example of the medical device assembly 600.

As shown in FIG. 6, surfaces of the antimicrobial device 400 may at least temporarily directly physically contact surfaces of the medical device 500 at interfaces 602. As a non-limiting example, at least one of the inner sidewalls 412 (FIGS. 4 and 5) and the floor 414 (FIG. 5) of the housing structure 402 defining the opening 404 (FIGS. 4 and 5) within the antimicrobial device 400 may temporarily directly physically contact the outer surface 512 of the male connection structure 508 of the medical device 500 at one or more of the interfaces 602. The antimicrobial device 400 may substantially decontaminate (e.g., substantially disinfect, substantially sterilize, etc.) portion(s) of the medical device 500 in at least temporary direct physical contact therewith at the interfaces 602. The portion(s) of the medical device 500 (e.g., the outer surface 512 of the male connection structure 508) may be provided in direct physical contact with the antimicrobial device 400 for any amount of time (e.g., duration) sufficient to substantially decontaminate the portion(s) of the medical device 500 and/or to substantially protect the portion(s) of the medical device 500 from future contamination. The duration(s) of direct physical contact between the antimicrobial device 400 and the medical device 500 may be substantially similar to the duration(s) of direct physical contact between the antimicrobial device 100 and the medical device 200 previously described with respect to FIG. 3.

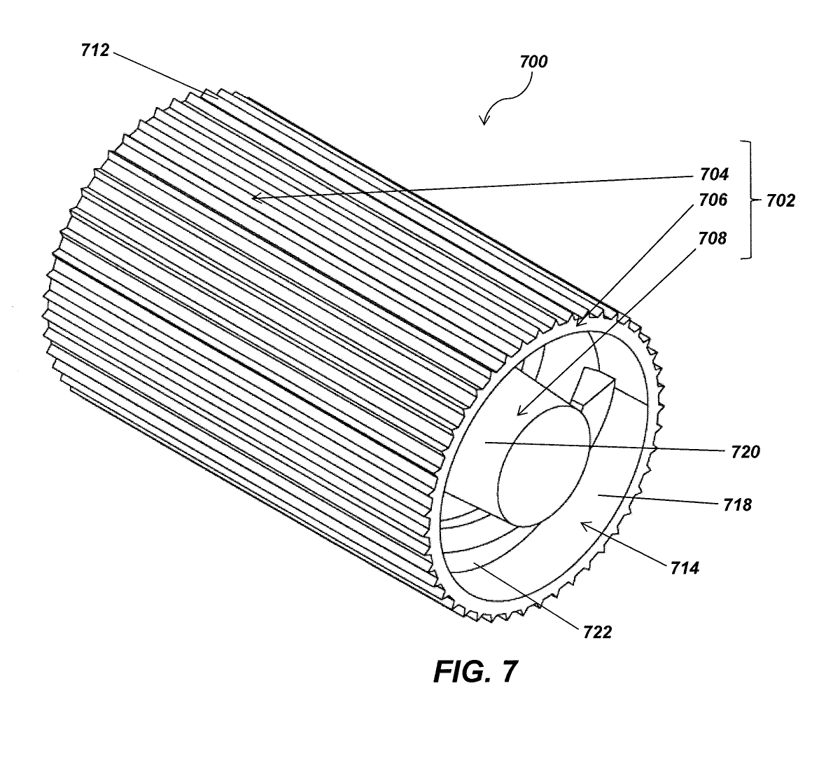

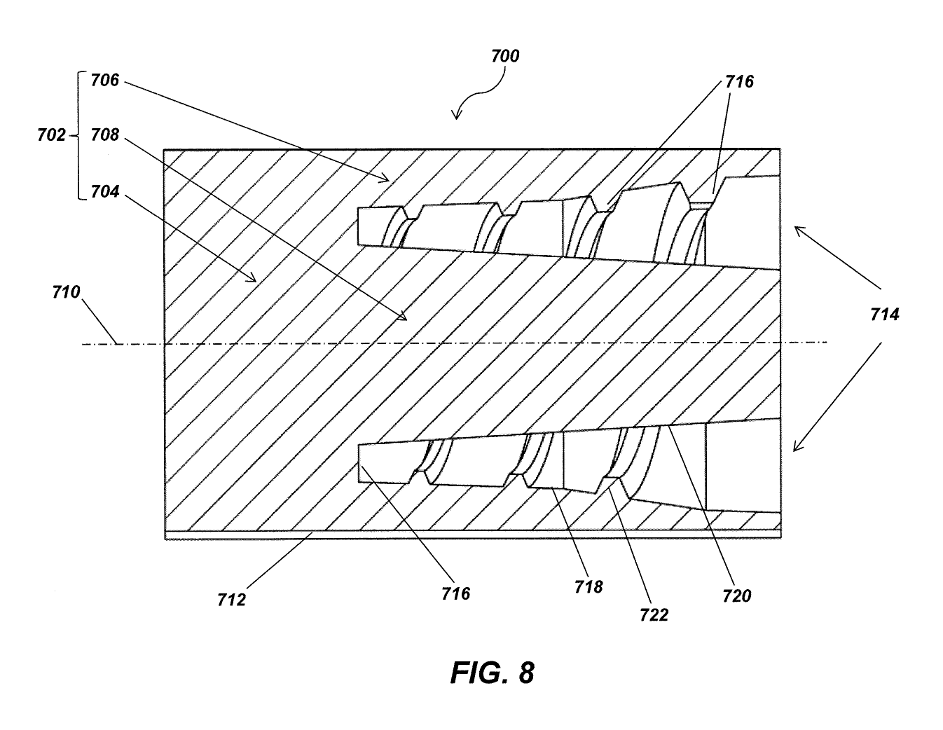

FIG. 7 illustrates a perspective view of an antimicrobial device 700 (e.g., antimicrobial cap, such as an antimicrobial medical device cleaning cap), in accordance with another embodiment of the disclosure. The antimicrobial device 700 is configured to attach to and decontaminate (e.g., disinfect, sterilize, clean, etc.) at least a portion of a medical device, as described in further detail below. FIG. 8 is a cross-sectional side view of the antimicrobial device 700 depicted in FIG. 7. While FIGS. 7 and 8 depict a particular configuration of the antimicrobial device 700, one of ordinary skill in the art will appreciate that the antimicrobial device 700 may exhibit a different configuration, such as a configuration exhibiting at least one of a different size, a different shape, different features, and different feature spacing. FIGS. 7 and 8 illustrate just one non-limiting example of the antimicrobial device 700.

As shown in FIGS. 7 and 8, the antimicrobial device 700 may include a housing structure 702 exhibiting a base region 704, a female connection region 706 longitudinally protruding (e.g., longitudinally projecting, longitudinally extending, etc.) from and integral with the base region 704, and a male connection region 708 longitudinally protruding from and integral with the base region 704 and at least partially (e.g., substantially) circumscribed by the female connection region 706. As used herein with respect to the antimicrobial device 700, each of the terms “longitudinal” and “vertical” means and includes extending in a direction substantially parallel to a central axis 710 (FIG. 8) of the antimicrobial device 700, regardless of the orientation of the antimicrobial device 700. Accordingly, as used herein with respect to the antimicrobial device 700, each of the terms “lateral” and “horizontal” means and includes extending in a direction substantially perpendicular (e.g., orthogonal) to the central axis 710 of the antimicrobial device 700, regardless of the orientation of the antimicrobial device 700. As described in further detail below, the female connection region 706 of the housing structure 702 may be configured (e.g., sized, shaped, etc.) to receive and retain a male connection structure of a medical device within an opening 714 at least partially extending through the antimicrobial device 700, and the male connection region 708 of the housing structure 702 may be configured to be received and retained within an another opening extending through the male connection structure of the medical device. Furthermore, as depicted in FIGS. 7 and 8, the housing structure 702 may, optionally, exhibit one or more ribs 712 extending (e.g., longitudinally extending) along a periphery thereof. The ribs 712, if present, may assist a user (e.g., a practitioner, a healthcare provider, etc.) in gripping and manipulating the antimicrobial device 700 during use and operation.

As shown in FIG. 8, in some embodiments, the opening 714 comprises a blind opening extending partially through and terminating within the housing structure 702. By way of non-limiting example, the opening 714 may extend completely through and at least partially define the female connection region 706 and the male connection region 708. and may terminate at the base region 704. In additional embodiments, the opening 714 comprises a through opening extending completely through the housing structure 702. The opening 714 may be configured (e.g., shaped and sized) to at least partially receive a male connection structure of a medical device to facilitate the temporary retention and at substantial decontamination (e.g., sterilization, disinfection, etc.) of the male connection structure, as described in further detail below. The opening 714 may, for example, be configured such that at least one of inner sidewalls 718 of the female connection region 706, outer sidewalls 720 of the male connection region 708, and a floor 716 (if present) of the housing structure 702 defining the opening 714 abut against (e.g., directly physically contact) portions of the male connection structure of the medical device. In some embodiments, the opening 714 exhibits an annular, tapered shape wherein the opening 714 laterally intervenes between the female connection region 706 and the male connection region 708, and wherein one end (e.g., the end proximate the floor 716) of the opening 714 has a relatively smaller area and/or a relatively smaller volume than another end (e.g., the end distal from the floor 716) of the opening 714. In additional embodiments, the opening 714 may exhibit a different shape, such as an annular, substantially non-tapered shape. As shown in FIGS. 7 and 8, at least a portion of the inner sidewalls 718 of the female connection region 706 of the housing structure 702 may, optionally, exhibit threads 722 for coupling with a male connection structure of a medical device. In additional embodiments, the threads 722 may be omitted (e.g., absent) from the inner sidewalls 718 of the female connection region 706 of the housing structure 702. In such embodiments, the inner sidewalls 718 of the female connection region 706 may, optionally, exhibit different means (e.g., one or more flexible protrusions, interference fit, surface friction between components, etc.) for coupling with a male connection structure of a given medical device. In further embodiments, the outer sidewalls 720 of the male connection region 708 of the housing structure 702 may, optionally, exhibit threads (and/or a different attachment means, such as flexible protrusions) for coupling with a female connection structure of a medical device (e.g., in manner similar to that in a manner similar to that discussed above in relation to the antimicrobial device 100 to maximize contact between the antimicrobial device 700 and the medical device).

In some embodiments, the housing structure 702 of the antimicrobial device 700 is configured to maximize the amount of contact between surfaces of the housing structure 702 and surfaces of a medical device connected thereto and/or received therein, in a manner similar to that previously discussed in relation to antimicrobial device 100. For example, the stem region 706 of the housing structure 702 of the antimicrobial device 700 may be configured to maximize contact between the stem region 706 and a corresponding structure of a medical device.

The material composition of the antimicrobial device 700 may be substantially similar to the material composition of the antimicrobial device 100 previously described with respect to FIGS. 1 and 2. For example, antimicrobial device 700 may be formed of and include at least one antimicrobial polymer material exhibiting antimicrobial properties sufficient to substantially decontaminate (e.g., substantially sterilize, substantially disinfect, etc.) at least a portion of a medical device in at least temporary physical contact with the antimicrobial device 700. The antimicrobial polymer material may be formed of and include at least one antimicrobial elastomer (e.g., polyurethane), and, optionally, at least one antimicrobial additive (e.g., a silver-based antimicrobial agent, a non-silver-based antimicrobial agent, combinations thereof, etc.). In some embodiments, the antimicrobial device 700 is formed of and includes polyurethane. In addition, the antimicrobial device 700 may be substantially homogeneous (e.g., the antimicrobial device 700 may consist of the antimicrobial elastomer, or the antimicrobial elastomer and the antimicrobial additive may be substantially uniformly distributed throughout the antimicrobial device 700), or may be heterogeneous (e.g., the antimicrobial elastomer and/or the antimicrobial additive may be non-uniformly distributed throughout the antimicrobial device 700). In some embodiments, the antimicrobial device 700 is substantially homogeneous, and is substantially free of antimicrobial additive(s) (e.g., the antimicrobial device 700 is formed of and includes at least one antimicrobial elastomer, such as polyurethane, but not at least one antimicrobial additive). In additional embodiments, the antimicrobial device 700 is substantially homogeneous, and is formed of and includes at least one antimicrobial elastomer (e.g., polyurethane) and at least one antimicrobial additive. In further embodiments, the antimicrobial device 700 is substantially heterogeneous, and is formed of and includes at least one antimicrobial elastomer (e.g., polyurethane) and at least one antimicrobial additive.

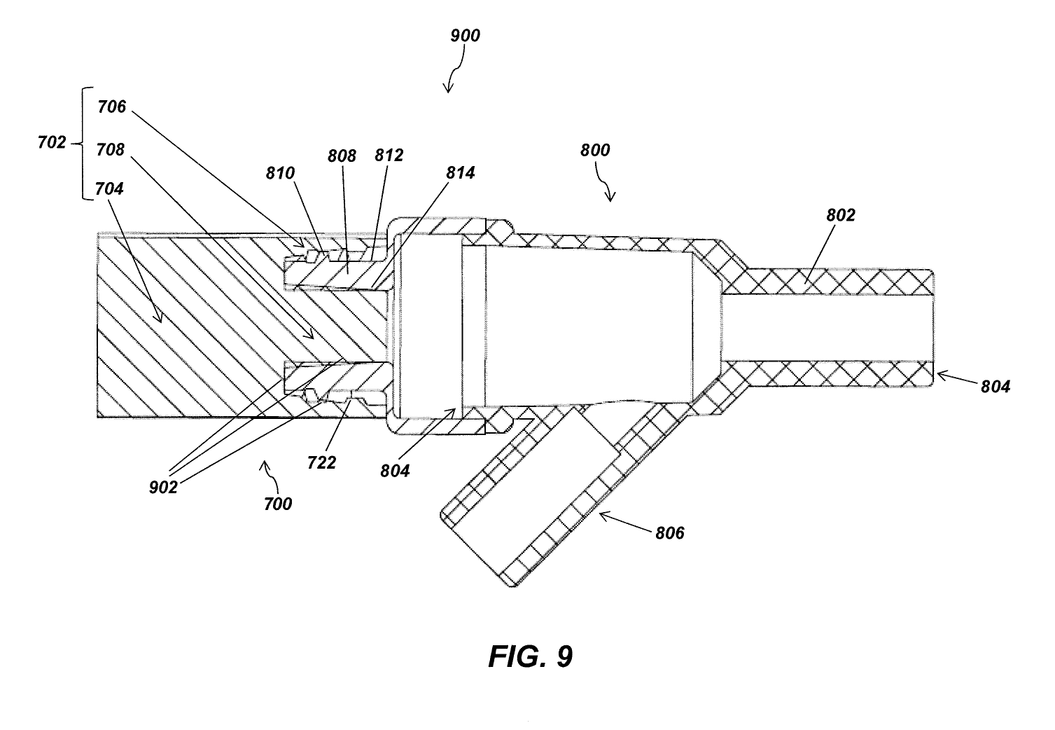

FIG. 9 is a cross-sectional side view of a medical device assembly 900, in accordance with an embodiment of the disclosure. The medical device assembly 900 includes a medical device 800 (e.g., needleless luer access port, Y-connector, etc.), and the antimicrobial device 700 previously described in relation to FIGS. 7 and 8. The antimicrobial device 700 may be coupled to the medical device 800. As shown in FIG. 9, the medical device 800 may include a housing structure 802 exhibiting opposing ends 804 and a branched region 806, and a connection structure 808 coupled to (e.g., attached to, bonded to, welded to, etc.) one of the opposing ends 804 of the housing structure 802. The housing structure 702 of the antimicrobial device 700 may be configured to receive and retain at least a portion of the connection structure 808 of the medical device 800 within the opening 714 (FIGS. 7 and 8) at least partially extending through the antimicrobial device 700. In addition, the male connection region 708 of the housing structure 702 of the antimicrobial device 700 may be configured to be received and retained within an opening extending through the connection structure 808 of the medical device 800. As shown in FIG. 9, the threads 722 projecting from the inner sidewalls 718 (FIGS. 7 and 8) of the female connection region 706 of the housing structure 702 of the antimicrobial device 700 may be configured (e.g., sized, shaped, spaced, etc.) to couple with additional threads 810 extending from an outer surface 812 of the connection structure 808 of the medical device 800. While FIG. 9 depicts a particular configuration of the medical device assembly 900, one of ordinary skill in the art will appreciate that different medical device assembly configurations are known in the art, which may be adapted to be employed in embodiments of the disclosure. FIG. 9 illustrates just one non-limiting example of the medical device assembly 900.

As shown in FIG. 9, surfaces of the antimicrobial device 700 may at least temporarily directly physically contact surfaces of the medical device 800 at interfaces 902. As a non-limiting example, at least one of the inner sidewalls 718 (FIGS. 7 and 8) of the female connection region 706, the outer sidewalls 720 (FIGS. 7 and 8) of the male connection region 708, and the floor 716 (FIG. 8) of the housing structure 702 defining the opening 714 (FIGS. 7 and 8) within the antimicrobial device 700 may at least temporarily directly physically contact at least one of the outer surface 812 and an inner surface 814 of the connection structure 808 of the medical device 800 at one or more of the interfaces 902. The antimicrobial device 700 may substantially decontaminate (e.g., substantially disinfect, substantially sterilize, etc.) portion(s) of the medical device 800 in at least temporary direct physical contact therewith at the interfaces 902. The portion(s) of the medical device 800 (e.g., the outer surface 812 of the connection structure 808, the inner surface 814 of the connection structure 808, etc.) may be provided in direct physical contact with the antimicrobial device 700 for any amount of time (e.g., duration) sufficient to substantially decontaminate the portion(s) of the medical device 800 and/or to substantially protect the portion(s) of the medical device 800 from future contamination. The duration(s) of direct physical contact between the antimicrobial device 700 and the medical device 800 may be substantially similar to the duration(s) of direct physical contact between the antimicrobial device 100 and the medical device 200 previously described with respect to FIG. 3.

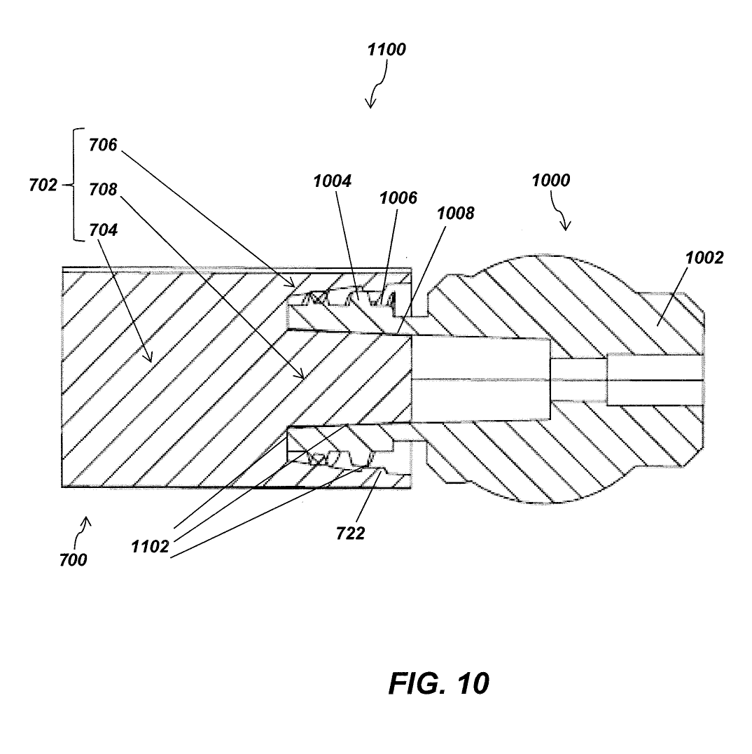

FIG. 10 is a cross-sectional side view of a medical device assembly 1100, in accordance with an embodiment of the disclosure. The medical device assembly 1100 includes a medical device 1000 (e.g., luer), and the antimicrobial device 700 previously described in relation to FIGS. 7 and 8. The antimicrobial device 700 may be coupled to the medical device 1000. As shown in FIG. 10, the medical device 1000 may include a female connection structure 1002 (e.g., a female luer structure). The housing structure 702 of the antimicrobial device 700 may be configured to receive and retain at least a portion of the female connection structure 1002 of the medical device 1000 within the opening 714 (FIGS. 7 and 8) at least partially extending through the antimicrobial device 700. In addition, the male connection region 708 of the housing structure 702 of the antimicrobial device 700 may be configured to be received and retained within an opening extending through the female connection structure 1002 of the medical device 1000. As shown in FIG. 10, the threads 722 projecting from the inner sidewalls 718 (FIGS. 7 and 8) of the female connection region 706 of the housing structure 702 of the antimicrobial device 700 may be configured (e.g., sized, shaped, spaced, etc.) to couple with additional threads 1004 extending from an outer surface 1006 of the female connection structure 1002 of the medical device 1000. While FIG. 10 depicts a particular configuration of the medical device assembly 1100, one of ordinary skill in the art will appreciate that different medical device assembly configurations are known in the art which may be adapted to be employed in embodiments of the disclosure. FIG. 10 illustrates just one non-limiting example of the medical device assembly 1100.

As shown in FIG. 10, surfaces of the antimicrobial device 700 may at least temporarily directly physically contact surfaces of the medical device 1000 at interfaces 1102. As a non-limiting example, at least one of the inner sidewalls 718 (FIGS. 7 and 8) of the female connection region 706, the outer sidewalls 720 (FIGS. 7 and 8) of the male connection region 708, and the floor 716 (FIG. 8) of the housing structure 702 defining the opening 714 (FIGS. 7 and 8) within the antimicrobial device 700 may at least temporarily directly physically contact at least one of the outer surface 1006 and an inner surface 1008 of the female connection structure 1002 of the medical device 1000 at one or more of the interfaces 1102. The antimicrobial device 700 may substantially decontaminate (e.g., substantially disinfect, substantially sterilize, etc.) portion(s) of the medical device 1000 in at least temporary direct physical contact therewith at the interfaces 1102. The portion(s) of the medical device 1000 (e.g., the outer surface 1006 of the female connection structure 1002, the inner surface 1008 of the female connection structure 1002, etc.) may be provided in direct physical contact with the antimicrobial device 700 for any amount of time (e.g., duration) sufficient to substantially decontaminate the portion(s) of the medical device 1000 and/or to substantially protect the portion(s) of the medical device 1000 from future contamination. The duration(s) of direct physical contact between the antimicrobial device 700 and the medical device 1000 may be substantially similar to the duration(s) of direct physical contact between the antimicrobial device 100 and the medical device 200 previously described with respect to FIG. 3.

In some embodiments, one or more of the antimicrobial device(s) of the disclosure (e.g., one of more of the antimicrobial devices 100, 400, 700) may initially be provided (e.g., packaged) in a disinfectant material (e.g., an alcohol, such as isopropyl alcohol and/or ethanol; hypochlorite; an oxygen reactive species, such as hydrogen peroxide; Triclosan; etc.). The disinfectant material may assist the antimicrobial device(s) with the rapid, at least partial decontamination of surfaces of one or more medical devices (e.g., one or more of the medical devices 200, 500, 800, 1000) in at least temporary physical contact with the antimicrobial device(s). Thereafter, the material properties of the antimicrobial device(s) may facilitate further decontamination of surfaces of the medical device(s), and/or may substantially protect the surfaces of the medical device(s) from future pathogenic contamination. In additional embodiments, one or more of the antimicrobial device(s) of the disclosure (e.g., one of more of the antimicrobial devices 100, 400, 700) may be brought into at least temporary physical contact with surfaces of one or more medical devices (e.g., one or more of the medical devices 200, 500, 800, 1000) without having been initially provided (e.g., packaged) in a disinfectant material.

The structures, devices, assemblies, and methods of the disclosure provide enhanced safety and improved simplicity as compared to structures, devices, assemblies, and methods conventionally associated with medical applications (e.g., material injection applications, material removal applications, etc.). For example, the antimicrobial devices 100, 400, 700 of the disclosure provide a simple and cost-effective means of substantially reducing the likelihood of pathogens entering the bloodstream of a subject (e.g., a patient) by way of a contaminated medical device configured to deliver fluid to the subject and/or to remove fluid from the subject. The antimicrobial devices 100, 400, 700 of the disclosure may be utilized with one or more portion(s) of a fluid flow system (e.g., a fluid delivery system), such as an intravenous (IV) device (e.g., an IV device including a peripheral IV line having a cannula in communication with vasculature of the subject, and exhibiting one or more connections, valves, and/or access ports) that delivers one or more fluid(s) to and from one or more blood vessels (e.g., veins) of the subject. The antimicrobial devices 100, 400, 700 of the disclosure may be provided in at least temporary (e.g., transient, for a selected period a time) physical contact with the one or more portion(s) of the fluid flow system to substantially decontaminate and/or protect the portion(s) of the fluid flow system that are exposed to potential contamination during use and operation (e.g., during use with a subject).

Once being apprised of the antimicrobial devices and medical device assemblies of the disclosure, one of ordinary skill in the art will be readily able to make and assemble the antimicrobial devices and the medical devices assemblies.

While embodiments of the disclosure may be susceptible to various modifications and alternative forms, specific embodiments have been shown by way of example in the drawings and have been described in detail herein. However, it should be understood that the disclosure is not limited to the particular forms disclosed. Rather, the disclosure encompasses all modifications, variations, combinations, and alternatives falling within the scope of the disclosure as defined by the following appended claims and their legal equivalents.

What is claimed is:

1. An antimicrobial device comprising a housing structure configured to couple to a medical device and comprising an antimicrobial polymer material comprising polyurethane, the antimicrobial polymer material formulated to at least partially decontaminate surfaces of the medical device in at least temporary physical contact with the housing structure.

2. The antimicrobial device of claim 1, wherein the antimicrobial polymer material comprises a substantially homogeneous distribution of the polyurethane.

3. The antimicrobial device of claim 1, wherein the antimicrobial polymer material comprises a substantially heterogeneous distribution of the polyurethane.

4. The antimicrobial device of claim 1, wherein the antimicrobial polymer material further comprises at least one antimicrobial additive.

5. The antimicrobial device of claim 4, wherein the at least one antimicrobial additive comprises at least one silver-based antimicrobial agent.

6. The antimicrobial device of claim 4, wherein the at least one antimicrobial additive comprises at least one non-silver-based antimicrobial agent.

7. The antimicrobial device of claim 1, wherein the antimicrobial polymer material is formulated to substantially disinfect the surfaces of the medical device in less than or equal to about 24 hours.

8. The antimicrobial device of claim 1, wherein the antimicrobial polymer material is formulated to substantially decontaminate the surfaces of medical device of one or more of Acinetobacter baumannii, Actinobacter spp, Aspergillus niger, Aspergillus flavus, Aureobasidium pullulans, Bacillus cereus, Bacillus thuringiensis, Bovine Viral Diarrhea Virus, Candida albicans, Chaetomium globosum, Citrobacter diversus, Cladosporium herbarium, Clostridium difficile, Enterobacter aerogines, Enterobacter cloacae, Enterobacteriaceae, Carbapenem-resistant Enterobacteriaceae, Enterococcus, Source and Rhinovirus and Vancomycin-resistant Enterococcus, Enterococcus faecalis, Epidennophyton floccosum, Escherichia coli, Gliocladtum vixens, Histoplasma capsulatum, Klebsiella oxytoca, Klebsiella pneumoniae, Legionella pneumpophila, Listeria monocytogenes, Mycobacterium tuberculosis, Penicillium funiculosum, Porphyromonas gingivalis, Proteus mirabilis, Proteus vulgaris, Pseudomonas aeruginosa, Rhizopus nigricans, S. saprophyticus, Saccharomyces cerevisiae, Salmonella gallinarum, Salmonella typhimurium, Stachybotrys, Staphylococcus aureus, Methicillin-resistant Staphylococcus aureus, Staphylococcus epidermidis, Streptococcus agalactiae, Streptococcus faecalis, Streptococcus pneumoniae, Streptococcus mutans, Trichophyton mentagrophytes, Trycophyton malmsten, and Vibrio parahaemolyticus.

9. The antimicrobial device of claim 1, wherein surfaces of the housing structure are configured to connect to the surfaces of the medical device and protect the surfaces of the medical device from future pathogenic contamination.

10. The antimicrobial device of claim 1, wherein the housing structure is configured to receive and retain at least a portion of a male connection structure of the medical device.

11. The antimicrobial device of claim 1, wherein at least a portion of the housing structure is configured to be received and retained within a female connection structure of the medical device.

12. A method of decontaminating a medical device, the method comprising coupling the medical device to an antimicrobial device comprising an antimicrobial polymer material comprising polyurethane, the antimicrobial polymer material formulated to at least partially decontaminate surfaces of the medical device in at least temporary physical contact with surfaces of the antimicrobial device.

13. The method of claim 12, wherein coupling the medical device to an antimicrobial device comprises directly physically contacting the surfaces of the medical device with the surfaces of the antimicrobial device.

14. The method of claim 12, further comprising selecting the antimicrobial device to exhibit a substantially homogeneous distribution of the polyurethane.

15. The method of claim 12, further comprising selecting the antimicrobial device to exhibit a substantially heterogeneous distribution of the polyurethane and at least one antimicrobial additive.

16. The method of claim 15, wherein selecting the antimicrobial device to exhibit a substantially heterogeneous distribution of the polyurethane and at least one antimicrobial additive comprises selecting the antimicrobial device to exhibit a relatively higher concentration of the at least one antimicrobial additive proximate the surfaces of the antimicrobial device.

17. The method of claim 12, wherein coupling the medical device to an antimicrobial device comprises coupling the medical device with the antimicrobial device for a period of time less than or equal to about 24 hours.

18. The method of claim 12, further comprising introducing the antimicrobial device to a disinfectant material prior to coupling the medical device to the antimicrobial device.

19. A medical device assembly comprising:
a medical device; and
an antimicrobial device coupled to the medical device and comprising an antimicrobial polymer material comprising polyurethane, the antimicrobial polymer material formulated to at least partially sterilize at least one surface of the medical device in physical contact with at least one surface of the antimicrobial device.

20. The medical device assembly of claim 19, wherein at least a portion of the medical device is retained within and directly physically contacts at least a portion of the antimicrobial device.

21. The medical device assembly of claim 19, wherein at least a portion of the medical device directly physically contacts and retains at least a portion of the antimicrobial device.
